(19) United States
(12) Patent Application Publication
(10) Pub. No.: US 2014/0357952 A1
(43) Pub. Date: Dec. 4, 2014

(54) VIDEO ENDOSCOPE HAVING AN ADJUSTABLE VIEWING DIRECTION

(71) Applicant: Olympus Winter & Ibe GMBH, Hamburg (DE)

(72) Inventors: Martin KROHN, Waren (DE); Jens ROSE, Hamburg (DE)

(73) Assignee: OLYMPUS WINTER & IBE GMBH, Hamburg (DE)

(21) Appl. No.: 14/462,705

(22) Filed: Aug. 19, 2014

Related U.S. Application Data
(63) Continuation of application No. PCT/EP2013/000413, filed on Feb. 13, 2013.

(30) Foreign Application Priority Data
Feb. 20, 2012 (DE) 102012202552.9

(51) Int. Cl. A61B 1/00; A61B 1/05

(52) U.S. Cl. CPC A61B 1/00183; A61B 1/051; A61B 1/00158; A61B 1/00066; A61B 1/0016; A61B 1/00006; A61B 1/00096
USPC 600/112

(57) ABSTRACT

A video endoscope having a handgrip and shaft having a cladding tube, wherein a prism unit having at least two prisms is connected to the cladding tube, wherein at least one distally arranged prism is rotated to modify a viewing angle around a rotation axis, an inner positioning system that comprises at least one rotational body arranged on a rotation axis of the shaft, axially fastened, and rotatable around the longitudinal axis of the shaft, to the distal tip of which the at least one image sensor is fastened, and at least one axially movable translational body, wherein a drive device is configured such that upon actuating a first control element, only the rotational body is rotated, and upon actuating a second control element, the translational body is moved and the rotational body is rotated such that a horizon position of an image formed on the image sensor remains constant.

### CROSS-REFERENCE TO RELATED APPLICATION

The present application is a continuation of PCT/EP2013/000413 filed on Feb. 13, 2013, which is based upon and claims the benefit to DE 10 2012 202 552.9 filed on Feb. 20, 2012, the entire contents of each of which are incorporated herein by reference.

### BACKGROUND

1. Field

The invention relates to a video endoscope having an adjustable viewing direction, a proximal handgrip and an endoscope shaft having a cladding tube that is connected to the handgrip in a rotationally fixed manner, wherein a prism unit having at least two prisms is connected to the cladding tube distally in the endoscope shaft in a rotationally fixed manner, wherein at least one image sensor is arranged proximally behind the prism unit, wherein at least one distally arranged prism of the prism unit can be rotated to modify a viewing angle about an axis of rotation crosswise to the longitudinal axis of the endoscope shaft, wherein the prism unit and the at least one image sensor are arranged in a hermetic chamber within the cladding tube, which extends out of the endoscope shaft into the handgrip.

2. Prior Art

Endoscopes, and particularly video endoscopes, with which the light from an operative field entering at a distal tip of an endoscope shaft of the endoscope is focused by an optical system onto one or more image sensors, are known in different embodiments. Thus, there are endoscopes with a direct view, a so-called 0° viewing direction, or endoscopes with lateral viewing direction, which have for example a lateral viewing direction of 30°, 45°, 70° or similar deviating from the 0° viewing direction. Here, the named angular degree means the angle between the central viewing axis and the longitudinal axis of the endoscope shaft. Further, there are endoscopes, or respectively video endoscopes, having an adjustable lateral viewing direction, with which the viewing angle, thus the deviation from the direct view, is adjustable.

Along with an adjustment of the viewing angle, thus the deviation from the direct view, the viewing direction, thus the azimuth angle, can also be adjusted about the longitudinal axis of the endoscope shaft, in that the endoscope as a whole is rotated about the longitudinal axis of the endoscope shaft.

Although with sideways viewing endoscopes typically the term “viewing direction” (direction of view, DOV) is used generally, in the following, in the scope of the present patent application and invention, a distinction is made between the “viewing direction”, which is to correspond to the azimuth angle of the rotation of the endoscope about the longitudinal axis of the endoscope shaft, and the “viewing angle”, which is to designate the polar angle, that is, the deviation from the direct view.

With video endoscopes, a change of the viewing direction, thus a rotation about the longitudinal axis of the endoscope shaft, represents a challenge insofar as in this case the image sensor of the video endoscope is also rotated, such that the horizon position or horizon orientation of the represented image changes. This can be corrected electronically, wherein then means for determining the actual horizon position must be present, for example gravity sensors. Another possibility is to mount the image sensor or image sensors rotatably in the video endoscope so that the horizon position can be corrected or maintained by a rotation of the image sensors in the video endoscope.

The European patent application EP 2 369 395 A1 shows an optical system for a video endoscope in which a change of the viewing angle is accomplished in that one prism of a prism unit having three prisms is rotated about a rotation axis that lies perpendicular, or respectively crosswise, to the longitudinal axis of the endoscope shaft. The other two prisms, which, together with the first prism, define the optical path, are not rotated along with the first prism such that the reflection surface of the first prism, which is rotated, is rotated relative to the corresponding reflection surfaces of the second prism. This results in a change of the horizon position of the displayed image. For this purpose, it is proposed in EP 2 369 395 A1 that a rotation of the image sensor should accompany the pivoting of the first prism. To this end, the image sensor is arranged in a rotatable tube. The prism unit is retained at a tube distal to this tube, wherein the two tubes can rotate relative to each other. A flexible section of the endoscope shaft adjoins the rotatable tube with the image sensor.

### SUMMARY

Based on this prior art, an object of the present invention is to specify a video endoscope having an adjustable viewing direction, with which the horizon position can be maintained during a change of viewing angle and a change of the viewing direction in a simple manner, wherein the video endoscope in addition should be autoclavable.

This object is solved by a video endoscope having an adjustable viewing direction, a proximal handgrip and an endoscope shaft having a cladding tube that is connected to the handgrip in a rotationally fixed manner, wherein a prism unit having at least two prisms is connected to the cladding tube distally in the endoscope shaft in a rotationally fixed manner, wherein at least one image sensor is arranged proximally behind the prism unit, wherein at least one distally arranged prism of the prism unit can be rotated about a rotation axis crosswise to the longitudinal axis of the endoscope shaft to modify a viewing angle, wherein the prism unit and the at least one image sensor are arranged in a hermetic chamber within the cladding tube that extends out of the endoscope shaft into the handgrip, that is further developed in that a first control element, for setting a horizon position of an acquired image, and a second control element, for setting the viewing angle of the rotatable prism, are arranged outside of the hermetic chamber, wherein arranged in the hermetic chamber there is an inner positioning system, which comprises at least one rotational body, which is arranged on a central rotation axis of the endoscope shaft, axially fastened, and rotatable about the longitudinal axis of the endoscope shaft, to the distal tip of which at least one image sensor is fastened, and at least one axially movable translational body, wherein the translational body is connected in a distal end section of the endoscope shaft to a gear mechanism that converts a translational movement of the translational body into a rotation of the at least one rotatable prism, wherein a drive device, which comprises the first control element and the second control element, is comprised and designed to move the rotational body and the translational body, wherein a drive device is designed such that upon actuating the first control element, only the rotational body is rotated, and upon actuating the second control element, the translational body is moved and the rotational body is rotated such that a horizon position of an image formed on the at least one image sensor remains constant.

According to the invention, the change of the viewing direction and the change of the viewing angle are conveyed using a translational body and a rotational body, wherein the translational body is responsible for the change of viewing direction, because during a translation the translational body interacts with the distal prism in the distal end region and causes it to rotate. The rotational body supports the image sensor(s) and is responsible for the rotation thereof with the goal of a constant horizon position of the displayed image.

One or more lenses can also be arranged between the prism unit and the at least one image sensor.

The drive device which is provided with the video endoscope according to the invention synchronizes the rotation of the rotational body and the translation of the translational body such that in each case, with a change of the viewing angle as well as with a change of the viewing direction, thus on the one hand with a change of the viewing angle relative to the longitudinal axis of the endoscope shaft, and on the other hand with a change of the azimuth position, or respectively the azimuth angle, with a rotation about the longitudinal axis, the horizon position of the displayed image is maintained.

This entails a difference between a change of the viewing angle, for which both the translational body and the rotational body are moved, and a change of the viewing direction with which only the rotational body is moved.

Because the image sensor(s) according to the invention are arranged at the distal end of the rotational body, it is no longer necessary to effect a rotation of a cladding tube or a tube section. Therefore it is also possible to reach with a translational body up to the distal end of the endoscope, in the region in which the distal prism is arranged in the prism unit. This was not possible with an arrangement in which the image sensor unit is held in a separate rotatable tube section, without penetrating the hermetic closing of the hermetic chamber by all components therein.

Thus, the video endoscope according to the invention is significantly better suited also for the disinfection procedure using autoclaves, because the sensitive inner positioning system is in the interior of the hermetic chamber and thus is not affected by autoclaves.

The drive device preferably comprises at least one magnetic coupling for transferring a rotation from outside the hermetic chamber to the rotational body. The magnetic coupling comprises at least one external magnet and one internal magnet. The external magnet is formed outside of the hermetic chamber and is connected to the handgrip for example. The magnetic coupling also has another internal magnet ring in the hermetic chamber. The internal magnet ring is connected directly or indirectly to the rotational body such that a rotation of the part, for example the handgrip of the video endoscope, which is connected to the external magnet ring of the magnetic coupling, leads to a corresponding rotation of the rotational body. Thus, a reference system is produced for the position of the image sensor, or respectively image sensors, relative to the video endoscope as such, without limiting the mobility of the image sensor in the video endoscope.

The drive device also advantageously comprises at least one magnetic coupling for transferring an axial movement and/or a rotation about the longitudinal axis of the translational body from outside of the hermetic chamber to the translational body. The corresponding magnetic coupling also has an internal and an external magnet ring, which are each arranged in the hermetic chamber, or respectively outside of the hermetic chamber. The magnetic rings, or respectively pole shoes, are designed such that a force transfer is possible and thus also a movement in the axial and/or circumferential direction as a rotation. Thus, either due to a sliding movement of the external magnet ring the inner magnet ring and with it the translational body are entrained, and thus are slid, or a rotation of the external magnet ring is converted in the interior into a translational movement of the translational body. Likewise, the translational body can also be entrained rotatingly as such.

The magnetic couplings, which can be used alternatively or cumulatively to each other, provide a force transfer in a direct manner from outside of the hermetic chamber into the hermetic chamber without having to penetrate the hermetic chamber for this purpose.

It is further advantageous that an electrically driven motor, which in the active state causes a rotation of the rotational body, is arranged on an internal magnet support of the magnetic coupling acting on the rotational body. The electrically driven motor in this case is located on the internal magnet support, while the rotational body can in turn be rotated with respect to the internal magnet support. As the magnetic coupling rotates, the internal magnet support rotates with it. A further operation of the corresponding control element leads to an activation of the electrically driven motor, for example an electric motor, a linear motor, a piezomotor, an actuator or similarly suitable drive, and leads to a rotation of the rotational body with respect to the internal magnet support. In this manner, the reference frame, which is positioned in the magnetic coupling by the internal magnet support, is effectively separated from the actual rotation of the rotational body for the purpose of horizon tracking. The transfer of the action of the electrically driven motor to the rotational body can occur using gear wheels, a worm gear or similar.

Also advantageously, an electrically driven motor, which in the active state causes an axial movement of the translational body, is arranged on an internal magnet support of the magnetic coupling acting on the translational body. In this case, the magnetic coupling is expediently designed such that only a transfer of the rotation about the longitudinal axis of the translational body is caused. The translational movement is caused by an electrically driven motor, which can be an electric motor, a linear drive, a piezomotor, an actuator or a similarly suitable motor. The transfer can occur using a toothed gear drive, a worm gear or similar.

If advantageously, the two electrically driven motors can be actuated synchronized or are controlled via an electronic control device, then an effective control of both the viewing direction and the viewing angle of the video endoscope is possible while simultaneously maintaining the horizon position of the reproduced image.

Alternatively to an electronic synchronization, preferably and advantageously, a synchronization drive is comprised having a first gear drive part connected to the translational body or integral with the translational body, and having a second gear drive part connected in a rotationally fixed manner to or integral with the rotational body, wherein the second gear drive part comprises a substantially cylindrical body having a circumferential groove forming a section of a helical line or a thread, in which a projecting part or a thread of the first gear drive part engages. The synchronization drive ensures in the case of force applied from a single source of force, for example an electric motor or a mechanically, in particular hand-operated, control element, that the rotation of the rotational body and the translation of the translational body, and thus the desired setting of the viewing direction and the viewing angle, occur while maintaining the horizon position of the reproduced image. Here, neither two electric motors, nor two unsynchronized mechanical sources of force are required. The synchronization occurs using the synchronization drive.

The synchronization drive comprises two gear drive parts, which are in engagement with each other such that a translation of the translational body leads to a rotation of the rotational body, which is moved due to the circumferential groove forming section of a helical line, or respectively the thread, and the corresponding engagement of the first gear drive part.

Advantageously, the second control element is formed as a slide switch or as a lever, which is connected via a conversion means, in particular a gear mechanism or a lever mechanism, to a retainer, which is translationally movable in the axial direction of the endoscope shaft, and in which an external magnet of the axially movable magnetic coupling is mounted. This design outside of the hermetic chamber permits an effective transfer of translational movements into the hermetic chamber via an axially acting magnetic coupling. A conversion of a movement can be realized in a mechanically simple and reliable manner both with a gear drive, and also with a lever mechanism, wherein the reduction provides good control of the setting by the operator.

Preferably the first control element is designed as a rotating wheel, in particular with a contoured periphery, which has a larger radius, at least in sections, particularly in the circumferential direction, than the handgrip. Thus, the rotary wheel can be held fixed during an operation, such that solely by securely holding the rotary wheel as a first control element, the horizon position of the displayed image is always maintained, independently of whether a viewing direction and/or a viewing angle are changed.

In an advantageous further development, the translational body is designed as a translational tube and/or the rotational body is designed as a rotational tube. The design of the translational body as a translational tube and/or the rotational body as a rotational tube allows signal lines to be placed in the interior thereof. In addition, the rotational body for example can be arranged inside the translational body, without contact thereto.

Further characteristics of the invention will become apparent from the description of the embodiments according to the invention together with the claims and the included drawings. Embodiments according to the invention can fulfill individual characteristics or a combination of several characteristics.

### BRIEF DESCRIPTION OF THE DRAWINGS

The invention is described below, without restricting the general intent of the invention, based on exemplary embodiments in reference to the drawings, whereby we expressly refer to the drawings with regard to the disclosure of all details according to the invention that are not explained in greater detail in the text. The figures show:

FIG. 1 illustrates a schematic perspective representation of a video endoscope according to the invention,

FIG. 2 illustrates a schematic side view of a prism unit,

FIG. 3 illustrates a schematic top view of a prism unit,

FIG. 4 illustrates a schematic representation of a section through a drive device according to the invention,

FIG. 5 illustrates a schematic representation of a section through a further drive device according to the invention,

FIG. 6 illustrates a schematic cross-sectional representation of a section through an endoscope according to the invention,

FIG. 7 illustrates a schematic perspective representation of an external gear drive,

FIG. 8 illustrates a schematic perspective representation of a control element,

FIG. 9 illustrates a schematic representation of a section through an outer part of the drive device according to the invention,

FIG. 10 illustrates a schematic representation of a section through an inner part of the drive device according to the invention,

FIG. 11 illustrates a systematic perspective representation of a gear drive component, and

FIG. 12 illustrates a schematic perspective representation of an alternative drive device according to the invention.

### DETAILED DESCRIPTION

In the drawings, the same or similar types of elements and/or parts are provided with the same reference numbers so that a corresponding re-introduction can be omitted.

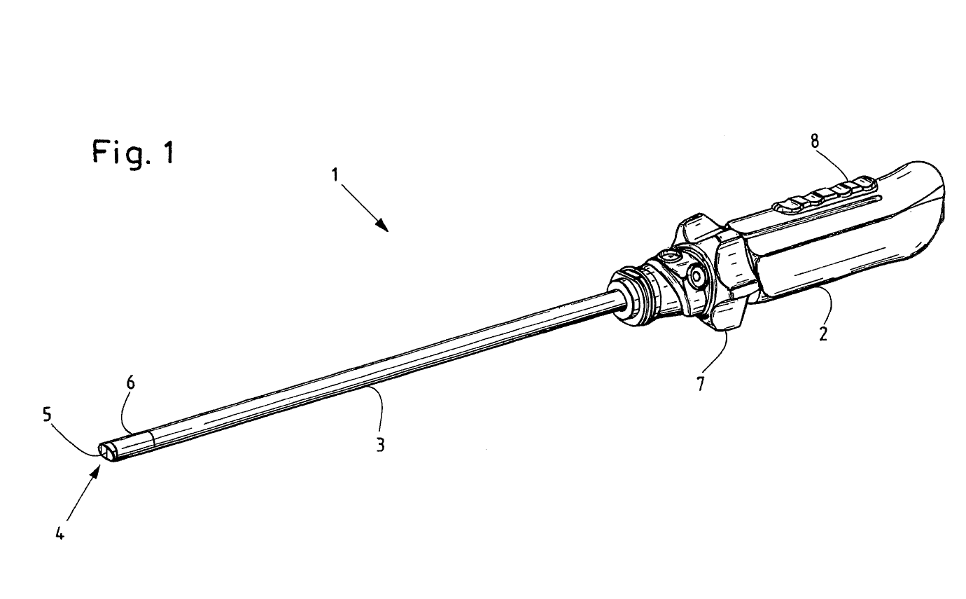

FIG. 1 shows a schematic perspective representation of a video endoscope 1 according to the invention having a proximal handgrip 2 and a rigid endoscope shaft 3. A viewing window 5 is arranged at the distal tip 4 of the endoscope shaft 3, behind which a distal section 6 of endoscope shaft is arranged having a prism unit, not shown, and an image sensor unit, not shown.

The viewing window 5 at the distal tip 4 is curved and asymmetrical. Thus, the viewing window 5 is formed to support a variable lateral viewing angle. A change in the viewing direction, thus a change of the azimuth angle about the longitudinal axis of the endoscope shaft 3, is effected by a rotation of the handgrip 2 about the central rotation axis, or respectively longitudinal axis of the endoscope shaft 3. The cladding tube of the endoscope shaft 3 is connected to the handgrip. The prism unit, not shown, at the distal tip 4 is also rotated with a rotation of the handgrip 2.

The handgrip 2 has a rotary wheel 7 formed as a first control element, and a second control element formed as a slide switch 8.

The rotary wheel 7 is held fixed during a rotation of the handgrip 2 for maintaining the horizon position of the displayed image. This has the effect that the image sensor in the interior of the endoscope shaft 3 does not perform the movement.

In order to change the viewing angle, thus the deviation of the viewing direction from the direct view, the slide switch 8 is moved. A distally directed sliding of the slide switch 8 leads, for example, to an increase of the viewing angle; a proximally directed return of the slide switch 8 in this case causes a reduction of the viewing angle up to the direct view. The actuation of the slide switch 8 is accompanied by a rotation of the image sensor, in order to maintain the horizon position of the displayed image even with a rotation of the prism unit.

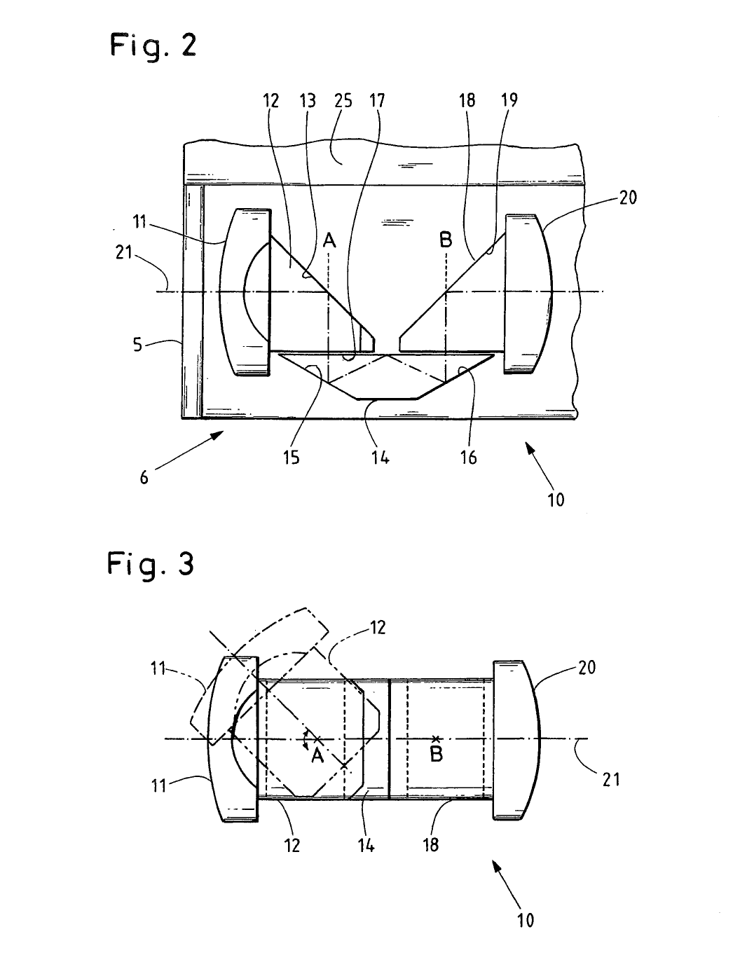

FIG. 2 shows a schematic side view of an appropriate prism unit 10. On the left side, light of a central optical path 21, which is shown as a dash dotted line, enters through a viewing window 5 and enters through an entry lens 11 into a first distal prism 12. The light hits a mirrored surface 13 and is reflected downward in the direction onto a second prism 14 and a mirrored surface 15 of the second prism. The mirrored surface 15 has an acute angle to the lower side 17 of the second prism 14 so that the central optical path is initially reflected on a central section of the lower side 17, that is also mirrored, and from there to a second mirrored surface 16 of the second prism 14. This second mirrored surface 16 also has an acute angle towards the lower side 17 so that the central optical path is again reflected upward (axis B). There, the light enters into a third prism 18 having a mirrored surface 19, through which the light of the central optical path 21 is reflected again centrally in a direction parallel to the longitudinal axis of the endoscope shaft 3, and exits the prism unit 10 there through an exit lens 20. Above the prism unit 10, also shown, is also a part of the fiber optic bundle 25, by means of which light is guided from the proximal to the distal tip in order to illuminate an otherwise dark operative field.

The first prism 12 can be rotated about the perpendicular axis A in order to adjust the lateral viewing angle. Thereby, the mirrored surfaces 13 and 15 also rotate counter to each other so that the horizon position of the image, that is further guided proximally, is changed during a rotation of the first prism 12 about the axis A. This must be compensated by a rotation of the image sensor or the image sensors.

FIG. 3 shows a schematic top view of the prism unit 10 from FIG. 2. The left side shows how the first prism 12 is arranged in a 0° viewing direction (solid line). Also shown with a dotted line, is that the first prism 12 together with the entry lens 11 is rotated about the rotation axis A. In this case, the overlapping region between the mirrored surface 13 of the first prism 12 and the mirrored surface 15 of the second prism 14 is rotated. The horizon position is accordingly also rotated.

Figuratively speaking, this rotation of the horizon can be explained as follows. If the prism unit 10 is arranged so that the rotation axis A in FIG. 2 is arranged upwards, thus perpendicular to the horizon, which is an imaginary horizontal line, this horizontal line is represented as a line at a height on the mirrored surface 13 of the prism 12. With a rotation of the first prism 12 about the rotation axis, this is independent of the rotation angle. The imaginary horizon, which is a horizontal line, remains a horizontal line on the mirrored surface 13. This imaginary horizontal line, if 0° is set as a viewing direction, as is shown in FIG. 3 with a solid line, is again depicted on a line on the first mirrored surface 15 of the second prism 14, which lies at a height, or respectively is arranged perpendicular to the longitudinal axis of the endoscope 1. When, as shown by the dotted lines in FIG. 3, the first prism 12 is rotated about the rotation axis A, the horizontal line also rotates on the mirrored surface 13, and thus rotates with respect to the mirrored surface 15 of the second prism 14. This horizontal line now runs across the mirrored surface 15, and thus is rotated. This must be compensated.

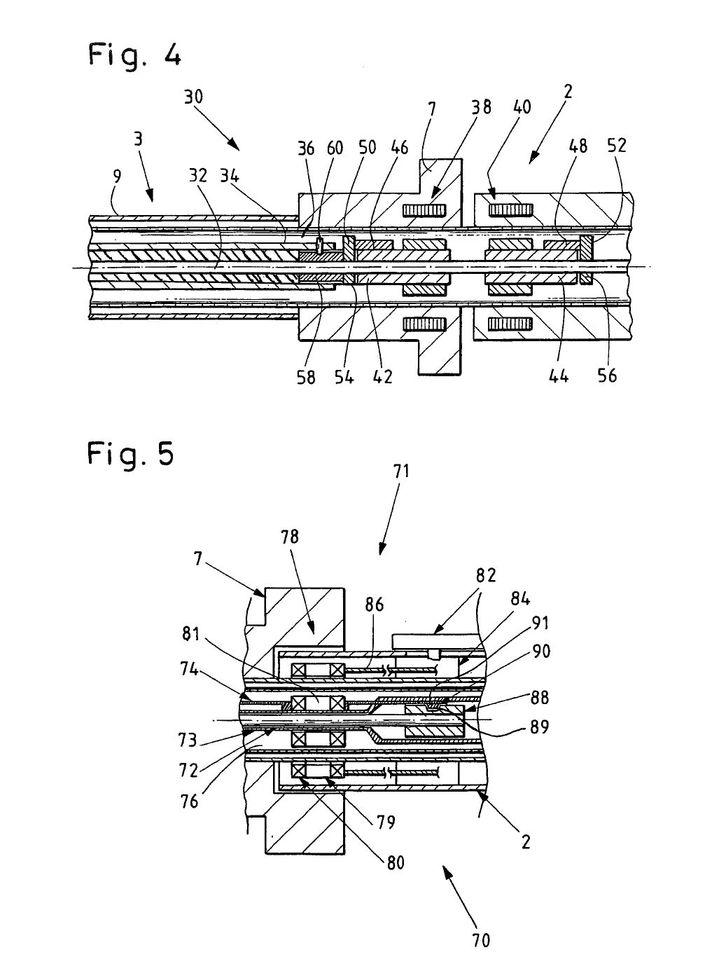

FIG. 4 shows in a schematic cross-section a first example embodiment of a drive device 30 according to the invention of a video endoscope according to the invention. This concerns the transition region between the handgrip 2 and the endoscope shaft 3. The handgrip 2 has a distal rotary wheel 7. The interior of the handgrip 2 and the rotary wheel 7, which is a part of the handgrip 2, are located in a hermetically closed chamber 36, which is embedded distally in the cladding tube 9 of the endoscope shaft 3, and extends also into the handgrip 2. A rotational body 32 and a translational body 34 are arranged centrally in the hermetic chamber 36. The rotational body 32 on the distal tip thereof, not shown, supports a unit having the image sensor(s), while the translational body 34 at the tip thereof, not shown, causes a rotation of the first prism from FIGS. 2 and 3.

The rotary wheel 7 is arranged rotatable with respect to the handgrip 2. The rotary wheel 7 comprises a magnetic coupling 38, which is formed so that a rotation of the rotary wheel 7 with respect to the handgrip 2, is transferred to an internal magnet ring of the magnetic coupling 38. This internal magnet ring is connected to a magnet support 42 in a rotationally fixed manner. An electric motor 46 that is attached to the magnet support 42, moves, via a gear wheel 50, a gear wheel 54 that is in connection with a groove body 58 having a oblique circumferential groove. The central rotational body 32 is mounted rotatably in the magnet support 42, as are the gear wheel 54 and the groove body 58.

A pin 60 ensures that the translational body 34 runs in the groove of the groove body 58, and thus a rotation of the groove body 58 leads to a translation of the translational body 34. At the same time, the magnet support 42 can be fixed to the rotary wheel 7, whereby a reference is set for the horizon position.

The viewing direction changes due to rotation of the handgrip 2. This influences the position of the distal prism unit, but not the position of the image sensor. In the handgrip 2 there is also a second magnetic coupling 40 with an external magnet ring and an internal magnet, by means of which in addition a rotation can be transferred to a second magnet support 44. A second electric motor 48 is arranged in a rotationally fixed manner on the magnet support 44; the motor in turn, via gear wheels 52 and 56, enables a rotation of the rotational body 32 in the magnet support 44 and the further components. This enables a reference of the horizon position.

A second control element, not shown in FIG. 4, can however be implemented as an electrical switch for setting the viewing angle, which via an electrical or electronic synchronization device causes the actuation of the two electric motors 46, 48.

The functioning of the drive device 30 from FIG. 4 is such that the motor 46 for changing the viewing angle moves the translational body 34 in the hermetic chamber 36, wherein the movement of the motor is converted by the gear wheels 50, 54. The motor 48 serves for tracking the image sensor(s) on the axial axis of the endoscope shaft 3 by rotating the rotational body 32. The two electric motors are located in each case on a magnet support 42, 44, the positions of which are conditioned by the magnetic couplings 38, 40, and the fields thereof, arranged on the rotary wheel 7 and the handgrip 2. The horizon position is changed by a rotation of the rotary wheel 7, wherein the motor 46, conditioned by the mounting on the magnet support 42, follows the movement of the rotary wheel 7.

FIG. 5 shows a schematic cross-section at representation of an alternative example embodiment without electric motors. The drive device 70 comprises a synchronization drive 71 that acts on a rotational body 72 and a translational body 74. The rotational body 72 is mounted in a bearing sleeve 73.

A slide control element 82 that is arranged on the handgrip 2, acts axially moving an external ring magnet 79 of a magnetic coupling 78, via a gear drive 84 and a slider 86. Thus, an axial movement is transferred to the internal ring magnet 81 of the magnetic coupling 78, thus into the hermetic chamber 76.

The internal ring magnet 81 is directly connected on one side to the translational body 74 so that an axial movement of the internal ring magnet 81 leads to a translational movement, thus sliding the translational body 74, which corresponds to a corresponding change of the viewing angle. On the other side, the internal ring magnet 79 is distally connected to a toothed rack 90, which has a catch 91 in the distal end region thereof which engages in a groove 89 of a groove support 88. The groove support 88 is a cylindrical body, having a circumferential groove 89 forming sections of helical lines, that is connected to the rotational body 72 in a rotationally fixed manner. A slide movement of the internal magnet ring 81 in the axial direction leads to a movement also of the catch 91, whereby the axially fixed rotational body is displaced in a corresponding rotation. Movement of the slide control element 82 leads therefore to a simultaneous change of the viewing angle, due to sliding of the translational body 74, and to a corresponding rotation of the image sensor, or respectively image sensors, due to a rotation of the rotational body 72.

If the slide control element 82 is not moved, a rotation of the handgrip 2 with respect to the rotary wheel 7 as a first control element still causes a rotation of the distal prism group, while in contrast, the translational body 74 and the rotational body 72 remain stationary and without rotation.

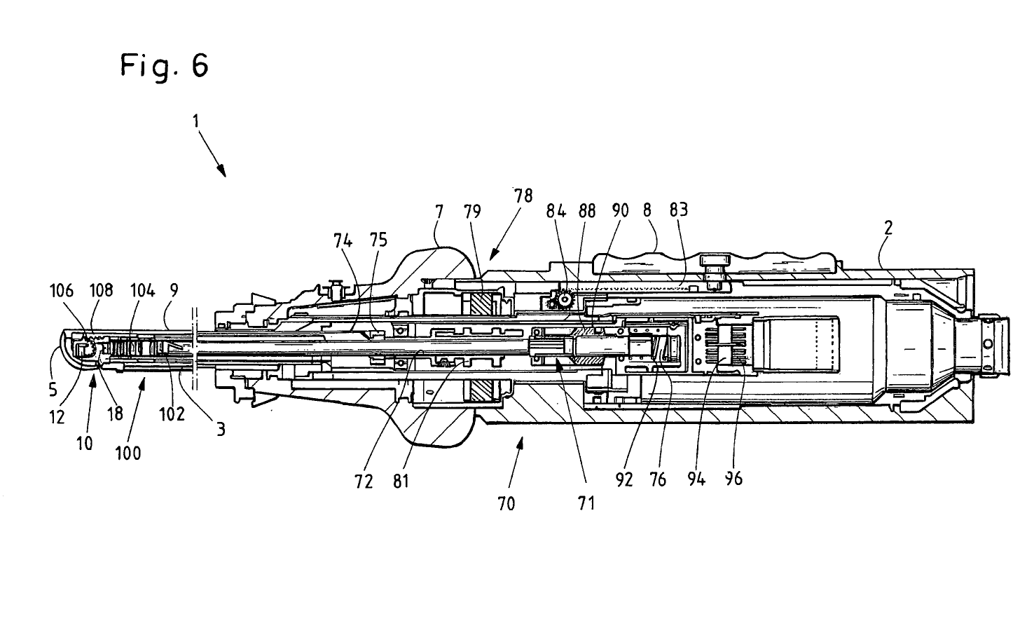

FIG. 6 shows a schematic cross-section of a video endoscope 1 according to the invention having a drive device 70. The drive device 70 corresponds substantially to that from FIG. 5.

In addition, FIG. 6 shows the distal region of the endoscope shaft 3 having a curved viewing window 5, behind which a first prism 12 of a prism group 10 is arranged, equipped with a gear wheel 106. The third prism 18 of the prism unit 10 is also shown, while the second prism 14 lies outside of the sectional plane. There is a toothed distal section 108 of the translational body 74 in engagement with the teeth of the gear wheel 106. An objective having lenses 104 adjoins proximally to the prisms unit 10 and thereon a sensor unit 100 having at least one image sensor 102. A plurality of image sensors can serve to improve the image quality, to create stereoscopic video images or to allow recording in different color regions.

The drive device 70 according to the invention having the synchronization drive 71 is located centrally in the central region of the handgrip 2. The handgrip 2 has a slide control element 8 and distally a rotary wheel 7. The rotary wheel 7 is connected to an external magnet 79 of a magnetic coupling 78, by means of which the horizon position of the video endoscope 1 is set. The internal magnet ring 81 of the magnetic coupling 78 is connected distally to a translational body 74 via a push connection 75, which also allows a rotation of the proximal region of the translational body 74 with respect to the distal region. The prism unit 10 can be rotationally decoupled from the magnetic coupling 78 in this manner. A rotational body 72 is rotationally mounted in the interior of the internal magnet ring 81. The rotational body on the distal tip thereof supports a sensor unit 100. The translational body 74 now runs outside of the rotational body 72 with respect to the central longitudinal axis of the endoscope shaft 3.

The rotational body 72 is connected proximally to a groove support 88, while the internal ring magnet 81 is connected proximally to a toothed rack 90 having a catch 91, which engages in a groove of the groove support 88. The groove support 88 is pre-loaded proximally from outside with a spring 92 so that the groove support 88 is axially fixed to the rotational body 72.

The hermetic chamber 76 is proximally hermetically sealed by a hermetic passage, in which contact pins are embedded, with which an electrical connection is possible to the outside of the hermetic chamber 76. The hermetic passage 94 can for example be a cast glass body having contact pins 96 molded therein.

Outside of the hermetic chamber 76 there is a gear drive 84, that on one side is in engagement with the slide control element 8, which is connected via a connection element to a connecting rod 83 having teeth, which by a movement of the slide control element 8 is also pushed in the axial direction of the endoscope shaft 3. The toothing of the connecting rod 83 is in engagement with a first gear wheel of the gear drive 84. The gear drive 84 converts this movement into a translational movement in the axial direction of the external magnet ring 79 of the magnetic coupling 78.

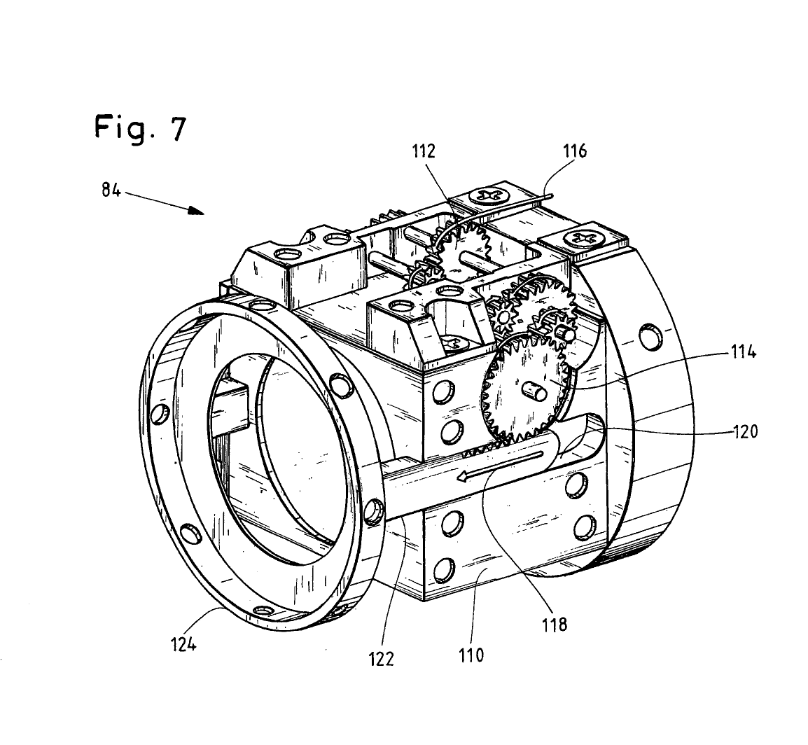

FIG. 7 shows a schematic perspective representation of the gear drive 84, or respectively the outer part thereof. The gear drive 84 comprises a gear drive body 110 having a central bore hole, into which the cladded pipe of the hermetic chamber 76 is inserted. The first gear wheel 112 is arranged centrally, or respectively in the center, and with a movement of the slide control element 8, shown in FIG. 6, in the direction of the arrow 116, is rotated in the corresponding direction. The further gear wheels of the gear drive 84 are provided with the appropriate direction of rotation arrows. A last gear wheel 114 of the gear drive is in engagement with teeth of a push arm 122, which is mounted axially movable in a groove 120 of the gear drive body 110. Here, the push arm 122 is pushed in the direction of arrow 118. Two symmetrical push arms 122 are provided that support a retainer 124, which retains and pushes an external ring magnet 79 of the magnetic coupling 78.

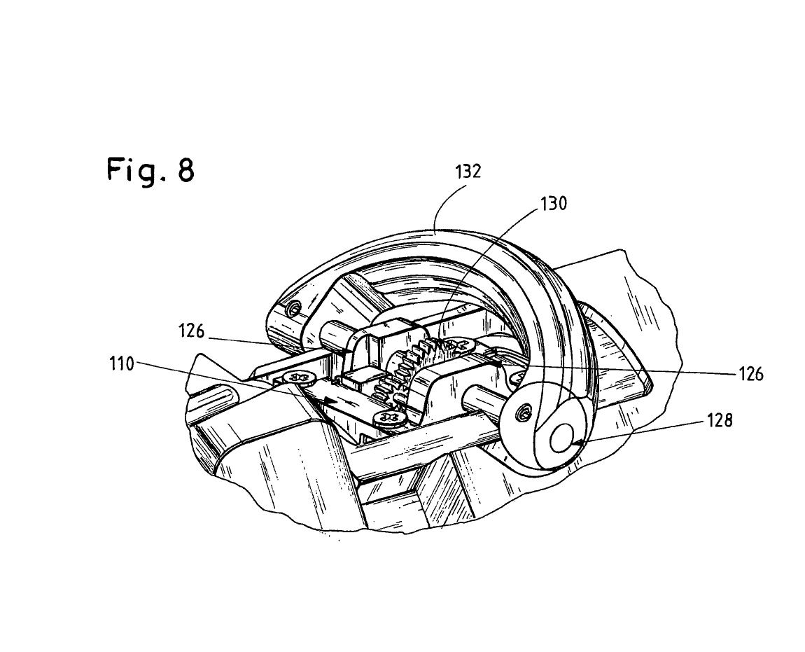

FIG. 8 schematically shows a section of an alternate control element variant. Here, the variant is a lever 132, or respectively a rocker arm, which is located on an axle 128, wherein by means of tilting the lever 132, the axle 128 also rotates. The axle 128 is mounted in two axle mounts 126. A first gear wheel 130 sits on the axle 128 and is in engagement with further gear wheels, as in FIG. 7, in order to effect a sliding of the push arm 122 in grooves 120 of the gear drive body 110. The viewing angle of the corresponding video endoscope is set in this manner.

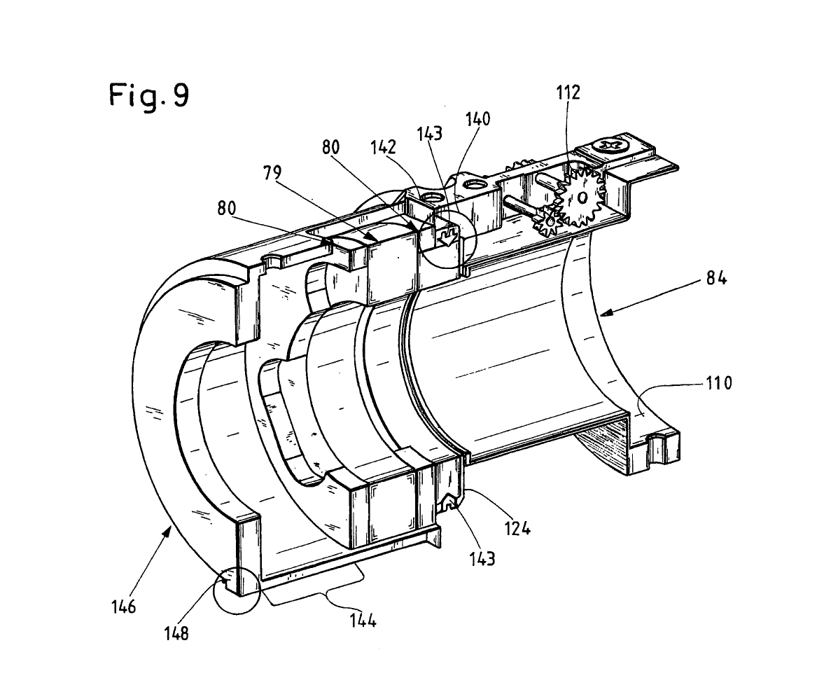

FIG. 9 shows the outer part of the drive device 70 in a schematic perspective outline and in cross-section. The gear drive 84 having the gear drive body 110, the first gear wheel 112, and the retainer 124 is shown proximally. In the retainer 124 there is a fixing arrangement 140 in which an adjustment ring 142 is fixed by means of fixing screws 143; the adjustment ring is joined distally by the external magnet ring 79 of the magnetic coupling 78 having a distal pole shoe 80 and a proximal pole shoe 80′. The external magnet ring 79 is mounted axially slidable, wherein a glide space 144 is provided for axially moving the magnetic coupling. The glide space 144 ends distally in a glide space connector 146, which additionally has a stopper 148 as a stop for limiting the azimuth rotation, that is, the viewing direction.

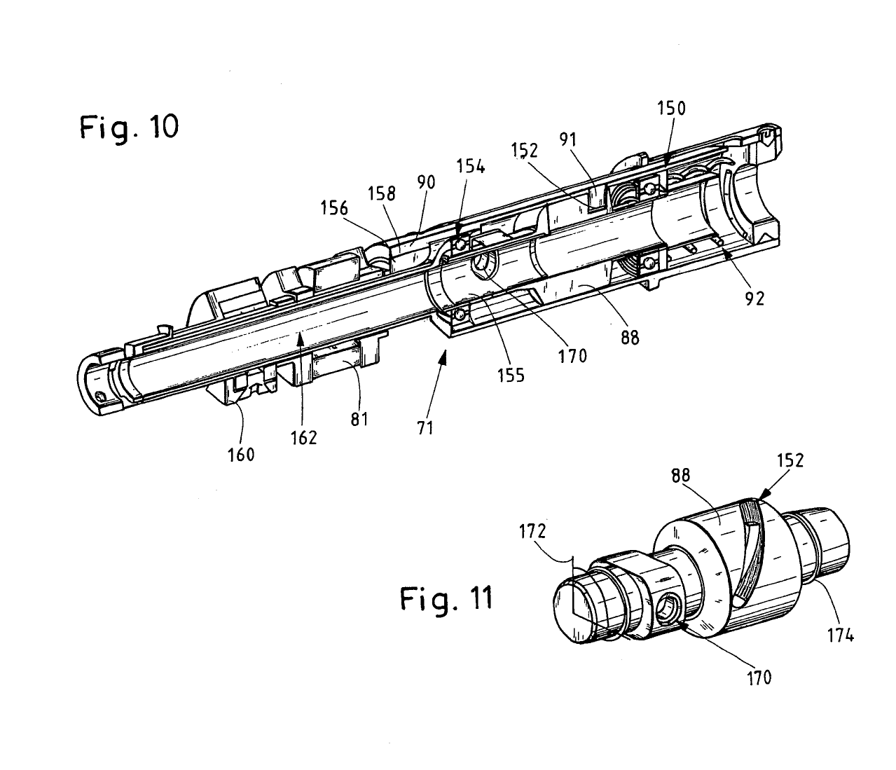

FIG. 10 shows the internal part, that is the part of the drive device 70 located in the hermetic chamber 76, in a schematic outline and in perspective representation. Centrally there is a groove body 88 having a groove 152 in operative engagement with the rotational body, of which a part 155 is shown. A bore hole 170 is provided in order to fix the rotational body with the part 155 thereof to the inside of the groove body 88. Proximally the groove body 88 is mounted in a bearing sleeve 150, and spring pressure is applied proximally by a compression spring 92 so that the groove body 88 and the rotational body are axially fixed. The groove body 88 is mounted distally rotatable in a ball bearing 154.

Outside of the groove body 88 there is a toothed rack 90 which has a catch 91, which engages in the groove 152 of the groove body 88. In the distal region, the toothed rack 90 has an inner contour which engages with an outer contour of a proximal push sleeve 156, which is connected axially movable to the internal ring magnet 81 of the magnetic coupling 78. With this, an axial movement of the ring magnet 81 leads to a corresponding axial movement of the proximal push sleeve 156 and the toothed rack 90, wherein the toothed rack 90 and the proximal push sleeve 156 are rotationally decoupled.

Distally, the internal ring magnet 81 is connected to a distal push sleeve 160, which further conducts the axial movement of the internal ring magnet 81 to a prism group, not shown.

In particular, the combination of the groove body 88 and the toothed rack 90 form a synchronization drive 71. In the interior of the rotational body there is a channel 162 in which electrical lines can be placed, for example.

FIG. 11 shows the groove body 88 from FIG. 10 in a perspective view. In a cylindrical part of the groove body 88 there is a groove 152, which delineates a quarter rotation about the circumference of the groove body 88. A widened region, arranged in a distal region of the groove body, has a bore hole 170 for fastening to the rotational body. Proximally, a stop ring 174 is provided on the groove body on which the spring 92 can press in order to axially fix the groove body 88 and the rotational body. FIG. 11 shows an angular region 172 at the distal end that corresponds to the angular region which can be set by means of the groove 152. The groove body 88 allows the rotational body and the image sensor(s) to rotate by 90°.

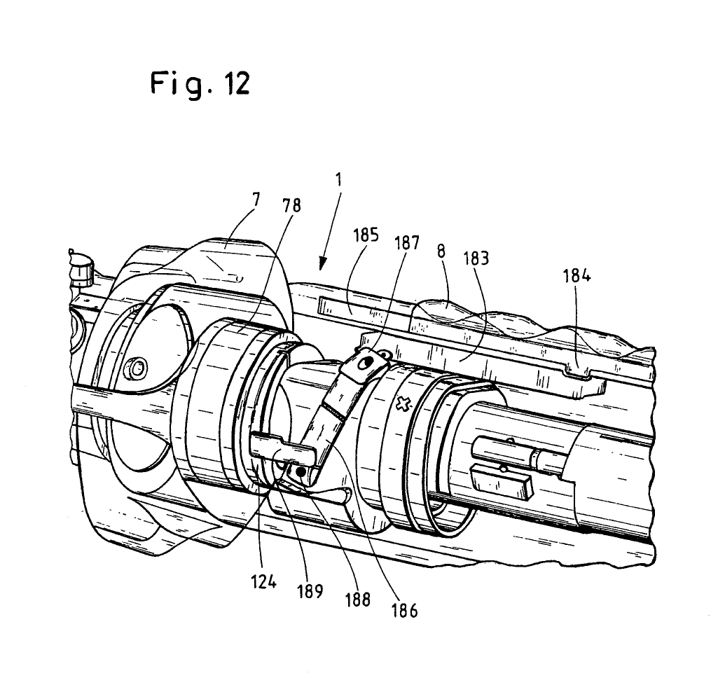

FIG. 12 shows an outline of a further example embodiment, in which in contrast to the example embodiment according to FIGS. 6 and 7, there is no gear wheel drive in order to transfer a movement of a slide switch 8 to a retainer 124 of a magnetic coupling 78, but rather a lever mechanism. For this purpose, the slide switch 8 has a pin 184 which engages in a corresponding recess of a connecting rod 183 guided in a rail 185. The connecting rod 183 engages at the distal end thereof in a coupling part 187 of a lever 186 of the lever mechanism, which is mounted pivotably on a peg 188 in the lower region. Somewhat above the peg 188, a slider 189 is attached to the lever 186, which is connected to the retainer 124 of the magnetic coupling 78. In this manner an axial movement of the slide switch 8 and the connecting rod 183 is converted into a smaller axial movement of the slider 189 according to the ratio of the lever arm of the lever mechanism. This embodiment is mechanically easier to implement and allows a very exact control of the magnetic coupling 78 with little or no play.

All named characteristics, including those taken from the drawings alone, and individual characteristics, which are disclosed in combination with other characteristics, are considered individually and in combination as essential to the invention. Embodiments according to the invention can be fulfilled through individual characteristics or a combination of several characteristics.

### REFERENCE LIST

1 video endoscope
2 handgrip
3 endoscope shaft
4 distal tip
5 viewing window
6 distal section
7 rotating wheel
8 sliding switch
9 cladding tube
10 prism unit
11 entry lens
12 first prism
13 mirrored surface
14 second prism
15, 16 mirrored surface
17 lower side
18 third prism
19 mirrored surface
20 exit lens
21 central optical path
25 fiber optic bundle
30 drive device
32 rotational body
34 translational body
36 hermetic chamber
38, 40 magnetic coupling
42, 44 magnet support
46, 48 electric motor
50, 52 gear wheel
54, 56 gear wheel
58 groove body
60 pin
70 drive device
71 synchronization drive
72 rotational body
73 bearing sleeve
74 translational body
75 push connection
76 hermetic chamber
78 magnetic coupling
79 external ring magnet
80, 80′ pole shoe
81 internal ring magnet
82 slide control element
83 connecting rod with teeth
84 gear drive
86 slider
88 groove support
89 groove
90 toothed rack with catch
91 catch
92 spring
94 hermetic penetration
96 contact pins
100 sensor unit
102 image sensor
104 objective with lenses
106 gear wheel
108 toothed section of a translational body
110 gear drive body
112 first gear wheel
114 last gear wheel
116, 118 push direction
120 groove
122 push arm
124 retainer
126 axle bearing
128 axle
130 first gear wheel
132 lever
140 fixing arrangement
142 adjustment ring
143 fixing screw
144 glide space
146 glide space connector
148 stopper
150 bearing sleeve
152 groove
154 ball bearing
155 part of the rotational body
156 proximal push sleeve
158 translation coupling
160 distal push sleeve
162 channel
170 bore hole
172 angular region
174 stop ring
183 connecting rod
184 pin
185 rail
186 lever
187 coupling part
188 peg
189 slider

What is claimed is:

1. A video endoscope having an adjustable viewing direction, the video endoscope comprising:
a proximal handgrip;
an endoscope shaft, the endoscope shaft having a cladding tube connected to the handgrip in a rotationally fixed manner, wherein in the endoscope shaft:
a prism unit having at least two prisms is connected distally to the cladding tube in a rotationally fixed manner;
at least one image sensor is arranged proximally behind the prism unit;
at least one distally arranged prism of the prism unit is rotated about a rotation axis crosswise to the longitudinal axis of the endoscope shaft to modify a viewing angle; and
the prism unit and the at least one image sensor are arranged in a hermetic chamber within the cladding tube that extends out of the endoscope shaft into the handgrip;
a first control element for setting a horizon position of an acquired image arranged outside of the hermetic chamber; and
a second control element for setting the viewing angle of the rotatable prism arranged outside of the hermetic chamber, wherein in the hermetic chamber an inner positioning system is arranged which comprises at least one rotational body arranged on a central rotation axis of the endoscope shaft, axially fastened, and rotatable about the longitudinal axis of the endoscope shaft, to the distal tip of which is fastened the at least one image sensor;
at least one axially movable translational body is connected in a distal end section of the endoscope shaft to a gear mechanism that converts a translational movement of the translational body into a rotation of the at least one rotatable prism; and
a drive device comprising a first control element and a second control element to move the rotational body and the translational body, wherein the drive device is configured such that upon actuating the first control element, only the rotational body is rotated, and upon actuating the second control element, the translational body is moved and the rotational body is rotated such that a horizon position of an image formed on the at least one image sensor remains constant.

2. The video endoscope according to claim 1, wherein the drive device comprises at least one magnetic coupling for transferring a rotation from outside of the hermetic chamber to the rotational body.

3. The video endoscope according to claim 1, wherein the drive device comprises at least one magnetic coupling for transferring an axial movement and/or a rotation about the longitudinal axis of the translational body from outside of the hermetic chamber to the translational body.

4. The video endoscope according to claim 2, further comprising an electrically driven motor is arranged on an internal magnet support of the magnetic coupling acting on the rotational body, and in the active state causes a rotation of the rotational body.

5. The video endoscope according to claim 2, further comprising an electrically driven motor is arranged on an internal magnet support of the magnetic coupling acting on the translational body, and in the active state causes an axial movement of the translational body.

6. The video endoscope according to claim 4, wherein the electrically driven motor is controlled via an electronic control device.

7. The video endoscope according to claim 5, wherein the electrically driven motor is controlled via an electronic control device.

8. The video endoscope according to claim 1, further comprising a synchronization drive having a first gear drive part connected to or integral with the translational body and having a second gear drive part connected in a rotationally fixed manner to or integral with the rotational body, wherein the second gear drive part comprises a substantially cylindrical body having a circumferential groove forming a section of a helical line or a thread, in which a projecting part or a thread of the first gear drive part engages.

9. The video endoscope according to claim 3, wherein the second control element is formed as one of a slide switch or a lever, which is connected via a conversion means to a retainer, which is translationally movable in the axial direction of the endoscope shaft, and in which an external magnet of the axially movable magnetic coupling is mounted.

10. The video endoscope according to claim 9, wherein the conversion means comprises one of a gear mechanism or a lever mechanism.

11. The video endoscope according to claim 1, wherein the first control element is formed as a rotary wheel.

12. The video endoscope according to claim 11, wherein the rotary wheel has a contoured periphery.

13. The video endoscope according to claim 11, wherein the rotary wheel has a larger radius at least in sections in the circumferential direction than the handgrip.

14. The video endoscope according to claim 1, wherein the translational body is formed as a translational tube.

15. The video endoscope according to claim 1, wherein the rotational body is formed as a rotational tube.
